(19) United States
(12) Patent Application Publication
(10) Pub. No.: US 2015/0257785 A1
(43) Pub. Date: Sep. 17, 2015

(54) Cuffed Fluid Line Extraction Device for Extraction of Tissue Ingrowth Cuffs and Fluid Lines from a Body and Associated Methods of Use

(71) Applicant: Shay Kent Seitz, Seal Beach, CA (US)

(72) Inventor: Shay Kent Seitz, Seal Beach, CA (US)

(21) Appl. No.: 14/205,367

(22) Filed: Mar. 11, 2014

(51) Int. Cl. A61B 17/34; A61M 25/01; A61M 25/10; A61B 17/3205

(52) U.S. Cl. CPC A61B 17/3468; A61B 17/32053; A61M 25/0194; A61M 25/10; A61M 2209/04

(57) ABSTRACT

A cuffed fluid line extraction device and method for facilitating the extraction of tissue ingrowth cuffs and fluid lines from a patient's body are provided. A cylindrical cuffed fluid line extraction device is disclosed, comprising a grip member located at the proximal end, and a beveled, “C-shaped,” smooth cutting head at the distal end that is spaced apart from the proximal end. An opening slit for insertion of the cuffed fluid line tube, in addition to a removal channel and channel guide, for receipt of a cuffed fluid line extends through and from the beveled, “C-shaped,” smooth cutting head at the distal end to and through the grip member at the proximal end. The “C-shaped,” smooth cutting head is designed as to support the cuffed fluid line during manipulation of the grip member as to allow the “C-shaped,” smooth cutting head beveled blades to cut away body tissue during extraction without severing of the fluid line.

### CROSS-REFERENCE TO RELATED APPLICATIONS

This application claims priority to U.S. Provisional Patent Application No. 61/794,428 filed on Mar. 15, 2013 titled CUFFED FLUID LINE EXTRACTION DEVICE FOR EXTRACTION OF TISSUE INGROWTH CUFFS AND FLUID LINES FROM A BODY AND ASSOCIATED METHODS OF USE, the disclosure of which is incorporated herein by reference.

### BACKGROUND OF THE INVENTION

The present invention relates to the field of medical devices used in the extraction of tissue ingrowth cuffs and fluid lines. More particularly, the invention relates to a circular-shaped cuffed fluid line extraction device with an opening slit for insertion of the cuffed fluid line tube and a removal channel for receiving the cuffed fluid line, facilitating the safe, convenient, and reliable manner for removal of cuffed or tunneled fluid lines.

Many medical procedures and therapies require the surgical implantation of a fluid line into a patient for removal or introduction of fluids. These procedures and therapies include introduction of, antibiotics, parenteral nutrition, IV fluids, blood products, peritoneal dialysis, and chemotherapy treatment; obtaining of blood samples; and preparation for plasmapheresis and hemodialysis. Such fluid lines can be inserted at specific entry points in the internal juglar, subclavian, or femoral veins, or intraperitoneal. These fluid lines are implanted into the patient for either short-term need, like surgery, or for extended periods associated with more long-term treatment applications.

Fluid lines include stabilizing devices for the critical purpose of anchoring the fluid line to the subcutaneous tissues of the patient when surgically implanted. There are many approaches for anchoring the fluid line to the patient; one of the most common approaches is the placement of a Dacron® fiber cuff on the implanted fluid line inside the body of the patient to promote tissue ingrowth within the fiber cuff. Most often, the fiber cuff is affixed around the fluid line tube. The tissue ingrowth cuff seals the opening created by the surgical implantation of the fluid line, from within the patient's body and helps stop bleeding and prevent infection. Although Dacron® cuffs are explicitly identified above; the fiber cuff can be made of any other biocompatible material.

Implantation of a fluid line involves surgically creating a subcutaneous tunnel formed by a tunneling tool, such as a trocar, through the skin and into the blood vessel of the patient, and positioning the cuff below the surface of the skin in the subcutaneous tissue. After a period of time, the surrounding tissue grows into the fabric of the cuff such that the fluid line is stabilized at the surgical entry point. While such fabric cuffs are inexpensive to make and are generally effective at stabilizing the fluid line, they are also difficult to remove. A common eventuality is that each and every fluid line cuff will need to be carefully extracted from the subcutaneous tunnel to allow for cuffed fluid line extraction from the patient's body.

In order to remove a fluid line from the ingrown subcutaneous tissue as a result of a tunnel, the medical practitioner must free the cuff from the subcutaneous tissue by cutting around the cuff into the subcutaneous tissue surrounding all edges of the cuff into which the tissue has grown with a scalpel or other cutting devices. Tissue growth occurs substantially transverse to the longitudinal axis of the cuff and around the outer circumference of the cuff. In addition, tissue growth occurs at angles oblique to the longitudinal axis of the cuff along both of the cuff's side edges. The medical practitioner must cut around the circumference of the cuff and both side edges to completely free the cuff and the indwelling fluid line. This surgical procedure is both difficult and time-consuming and can also result in increased patient bleeding and infection risk if the entire cuffed fluid line is not removed successfully. The procedure can also contribute to an increased chance of scar tissue formation and build-up within the subcutaneous tissues, which increases the difficulty of the surgical implantation and tunneling of future cuffed fluid lines. Also, since the fabric cuffs are generally sized as to provide a snug fit in the subcutaneous tissue, frictional forces against the tunnel wall make extraction through the tunnel extremely difficult.

There has been some effort to develop tools to make extraction of fluid lines with tissue ingrowth cuffs from patients easier. Such efforts can be seen in U.S. Patent Application No.'s 2006/0129134 A1 to Kerr, 2007/0185510 A1 to Tran, 2007/0244490 A1 to Moehle et. al., and U.S. Pat. No. 7,758,590 B2 to Daniele et. al. Despite the existence of such tools, there are still risks posed to patients undergoing these procedures, rendering improvements to fluid line extraction devices desirable and warranted. One such risk is the breaking or severing of the cuffed fluid line during extraction, which may require the medical practitioner to perform yet another surgical procedure to extract the severed distal portion of the cuffed fluid line that is left within the patient. This is caused in part due to the use of softer malleable materials, such as urethane and silicone in the production of fluid lines. Use of such materials in addition to the serrated and sharpened cutting edges of current tools increases the risk the fluid line will snap, break or become severed during the extraction procedure. Further, use of such materials also increases the risk the affixed cuff will result in the elongation of the fluid line tube and alter its dimensions while placing in traction. This stress placed on the proximal end of the fluid line during extraction from the tunnel is concentrated at the location of the cuff and can also lead to the snapping or breaking of the cuffed fluid line. Another risk to patients posed by the existing tools is the physical trauma caused to the skin and subcutaneous tissue due to the cumbersome nature and/or inferior cutting edge design. Use of existing tools for the extraction of cuffed fluid lines can result in patients sustaining unnecessary scaring, bruising and scar tissue formation. Lastly, existing tools are limited in their circumferential size and quality, often resulting in medical practitioners having to use the wrong size tool for the particular procedure.

With the foregoing in mind, a need exists for a removal method and device overcoming these shortcomings of the prior art. The present invention provides such an apparatus and associated method.

### BRIEF SUMMARY OF THE PRESENT INVENTION

The subject invention is directed to a cuffed fluid-line extraction device for the extraction of tissue ingrowth cuffs and fluid lines from the body. The device includes a grip member located at the proximal end of the cylindrical cuffed fluid line extraction device, extending until adjacent to the cutting shaft member and a razor sharp “C-shaped,” smooth cutting head at the distal end of the cutting shaft member. The grip member at the proximal end being spaced apart from the razor sharp “C-shaped,” smooth cutting head at the distal end by the grip member and cutting shaft member. An opening slit for insertion of the cuffed fluid line tube, in addition to a removal channel for receipt of a cuffed fluid line extends through and from the razor sharp “C-shaped,” smooth cutting head at the distal end to and through the grip member at the proximal end, passing through the cutting shaft member. The cuffed fluid-line extraction device includes a channel guide carried within the cuffed fluid line removal channel extending from the razor sharp “C-shaped,” smooth cutting head at the distal end, through the cutting shaft member and the grip member located at the proximal end, for supporting the cuffed fluid line during movement and manipulation of the grip member, allowing the razor sharp “C-shaped,” smooth cutting head to cut and dissect body tissue away from the tissue ingrowth cuff and fluid line for extraction of the cuffed fluid line from within the body.

For extraction of a cuffed fluid line, with tissue ingrowth, the razor sharp “C-shaped,” smooth, cutting head at the distal end shall have a circumference greater than the circumference of the tissue ingrowth cuff on the cuffed fluid line. The opening slit of the razor sharp “C-shaped,” smooth cutting head, cutting shaft member and grip member shall be greater than the diameter of the fluid line tube for insertion of the fluid line tube and receipt of the cuffed fluid line in the cuffed fluid line removal channel extending from the razor sharp “C-shaped,” smooth cutting head at the distal end through the cutting shaft member and the grip member located at the proximal end.

More particularly, the distal end razor sharp “C-shaped,” smooth cutting head may be beveled or tapered inward or outward and the cutting head shall be sharp and smooth to facilitate the safe cutting and dissection of body tissue.

Additionally, the cuffed fluid line extraction device may comprise a cutting shaft member connecting the distal end of grip member with the “C-shaped,” smooth cutting head.

Additionally, the cutting shaft member of the cuffed fluid line extraction device may comprise an open shaft with three equally spaced support members or one single, solid shaft connecting the distal end of the grip member through a retaining means and the razor sharp “C-shaped,” smooth cutting head at the distal end of the cutting shaft member.

The method for extracting a cuffed fluid line from a body may include providing a cuffed fluid line extraction device that includes a grip member located at the proximal end of the cylindrical cuffed fluid line extraction device, extending until adjacent to the cutting shaft member and a razor sharp “C-shaped,” smooth, cutting head at the distal end. Also an opening slit for insertion of the cuffed fluid line tube and a removal channel for receipt of a cuffed fluid line extending through and from the razor sharp “C-shaped,” smooth cutting head at the distal end to and through the grip member at the proximal end, passing through the cutting shaft member. Said opening slit of the razor sharp “C-shaped,” smooth cutting head, cutting shaft member and grip member shall be greater than the diameter of the fluid line tube for insertion of the fluid line tube and receipt of the cuffed fluid line in the cuffed fluid line removal channel extending from the razor sharp “C-shaped,” smooth cutting head at the distal end through the cutting shaft member and the grip member located at the proximal end. The cuffed fluid-line extraction device includes a cuffed fluid line channel guide carried within the fluid line removal channel and extending from the “C-shaped,” smooth cutting head at the distal end, through the cutting shaft member and the grip member located at the proximal end, for support of the cuffed fluid line during movement and manipulation of the grip member, allowing the “C-shaped,” smooth cutting head to cut and dissect body tissue away from the cuffed fluid line. After the body tissue has been separated from the cuffed fluid line, the fluid line is extracted from the patient's body.

More particularly, the distal end razor sharp “C-shaped,” smooth cutting head may be beveled or tapered inward or outward, the cutting head shall be sharp and smooth to facilitate the safe cutting and dissection of body tissue and shall have a circumference greater than that of the tissue ingrowth cuff on the cuffed fluid line.

Additionally, the method for removing a cuffed fluid line from a body may comprise a cuffed fluid line extraction device including a cutting shaft member connecting the distal end of grip member with the “C-shaped,” smooth cutting head. Said cutting shaft member of the cuffed fluid line extraction device may comprise an open shaft with three equally spaced support members or one single, solid shaft connecting the distal end of the grip member through a retaining means and the razor sharp “C-shaped,” smooth cutting head at the distal end of the cutting shaft member.

Other objects and advantages of the present invention will become apparent from the following detailed description when viewed in conjunction with the accompanying drawings, which set forth the certain embodiments of the invention.

### BRIEF DESCRIPTION OF THE DRAWINGS

FIG. 1 is a side perspective view of the present cuffed fluid line extraction device.

FIG. 2A is an enlarged sectional view showing the details of the cutting shaft member and beveled, “C-shaped,” smooth cutting head of the present cuffed fluid line extraction device, displaying the beveled, “C-shaped,” smooth cutting head manipulation and movement taken across the three-dimensional space B.

FIG. 2B is another sectional view showing the details of the cutting shaft member and beveled, “C-shaped,” smooth cutting head of the present cuffed fluid line extraction device.

FIG. 3 is an end view of the proximal end grip member of the present cuffed fluid line extraction device.

FIG. 4 is an end view of the distal end beveled, “C-shaped,” smooth cutting head of the present cuffed fluid line extraction device.

FIG. 5 is a bottom perspective view of the present cuffed fluid line extraction device.

FIG. 6 is a top perspective view of the present cuffed fluid line extraction device.

FIG. 7A is a longitudinal view of the present cuffed fluid line extraction device.

FIG. 7B is a cross-sectional view taken along section line B of FIG. 7A, displaying the beveled, “C-shaped,” smooth cutting head manipulation and movement taken across the three-dimensional space C.

FIGS. 7C and 7D are enlarged views displaying two variations of the “C-shaped,” smooth, beveled cutting head taken along section line B of FIG. 7A.

FIGS. 8A, 8B, and 8C are side views of a cuffed fluid line extraction device in accordance with the present invention before and during removal of a tissue ingrowth cuff and fluid line from a body, respectively.

FIG. 9 is a side perspective view of an alternative embodiment of the cuffed fluid line extraction device of FIG. 1.

FIG. 10A is an enlarged sectional view of an alternative embodiment of the cuffed fluid line extraction device of FIG. 2A, showing the details of the cutting shaft member and beveled, “C-shaped,” smooth cutting head of the present cuffed fluid line extraction device, displaying the beveled, “C-shaped,” smooth cutting head manipulation and movement taken across the three-dimensional space B.

FIG. 10B is another sectional view of an alternative embodiment of the cuffed fluid line extraction device of FIG. 2B, showing the details of the cutting shaft member and beveled, “C-shaped,” smooth cutting head of the present cuffed fluid line extraction device

### REFERENCE NUMERALS IN THE DRAWINGS

01 Cuffed Fluid Line Extraction Device
10 Grip Member
20 Cutting Shaft Member
30 “C-Shaped,” Smooth Cutting Head
40 Grip Member and Cutting Shaft Member Retaining Means.
50 Equally Spaced Cutting Shaft Member Connective Support Members, Connecting the “C-Shaped” Smooth Cutting Head at the Distal end to the Distal End of the Grip Member
50′ Alternative Embodiment of the Cutting Shaft Member Comprising a Single, Solid Shaft Connecting the “C-shaped,” Smooth Cutting Head at the Distal End to the Distal End of the Grip Member
80 Opening Slit
90 Cuffed Fluid Line Removal Channel
100 Cuffed Fluid Line Channel Guide
110, 110′ Alternative Embodiments for Rigid Gripping/Knuckling Structures Facilitating Gripping and Manipulation of Device by Hand or Instrument
120 “C-Shaped” Smooth Cutting Head Beveling Inward
120′ “C-Shaped” Smooth Cutting Head Blade Beveling Outward.

| | |
|---|---|
| 130 Human Body | 140 Cuffed Fluid Line |
| 150 Tissue Ingrown Cuff | 160 Longitudinal Axis |

### DETAILED DESCRIPTION OF THE PREFERRED EMBODIMENTS

The detailed embodiments of the present invention are disclosed herein and made with reference to the accompanying drawings. It should be understood, however, that the disclosed embodiments are merely exemplary of the invention, which may be embodied in various forms. Therefore, the descriptions disclosed herein should not be construed as limiting the embodiments set forth herein, rather, these embodiments are provided so that this disclosure will be thorough and complete. Like numbers refer to like elements throughout, and prime notation is used to indicate similar elements in alternative embodiments.

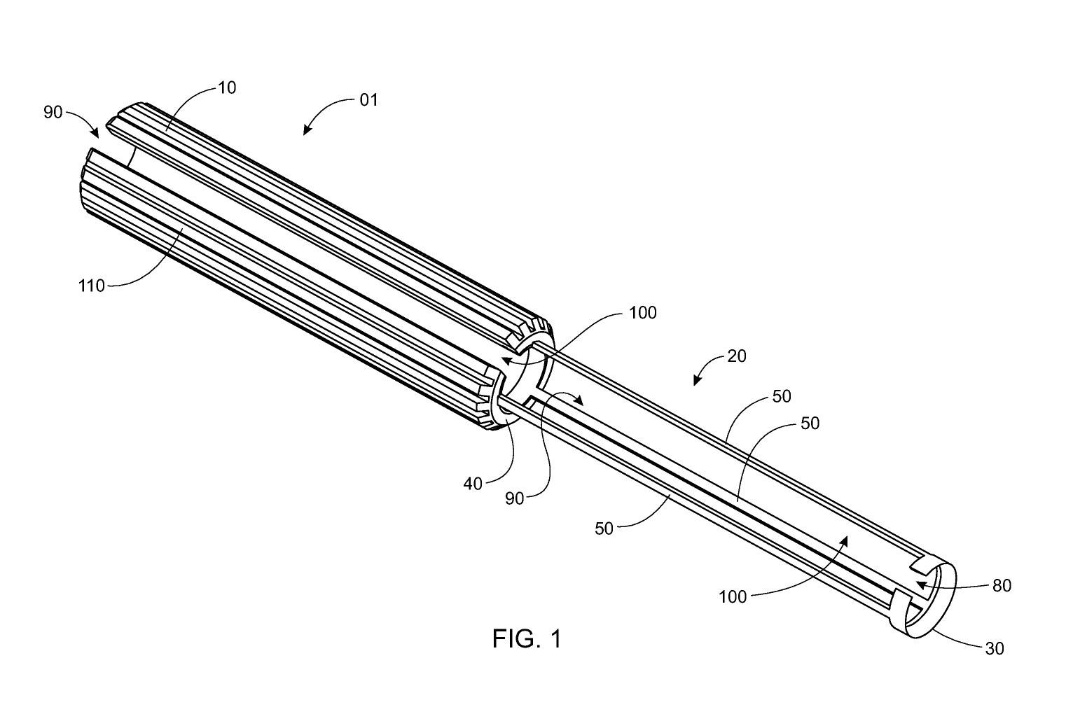

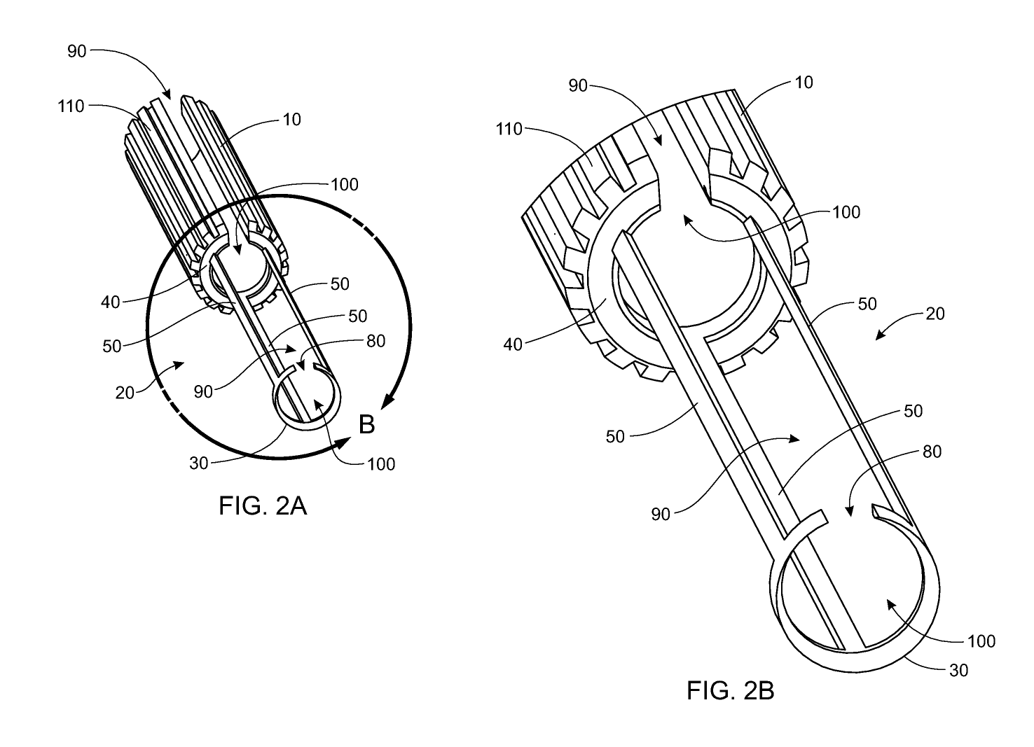

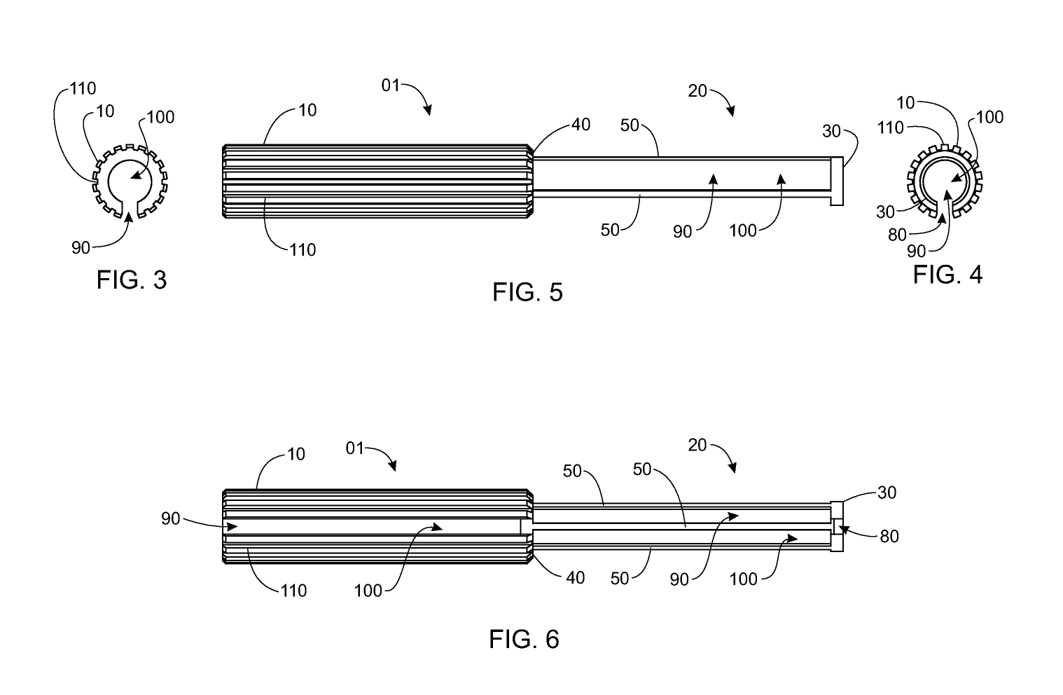

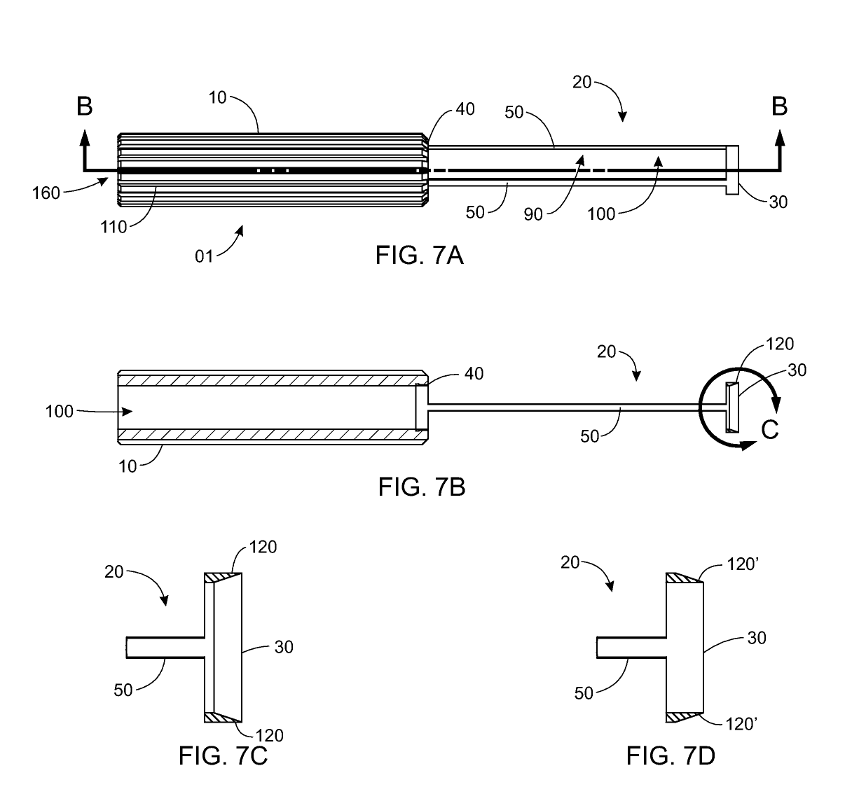

With reference to FIGS. 1 through 8C, a cuffed fluid line extraction device 01 is disclosed. The cuffed fluid line extraction device 01 is designed to cut or dissect the fibrous tissue from the tissue ingrown cuff 150 of a tunneled fluid line 140 in order to remove the tissue ingrowth cuff 150 and the fluid line 140 from the patient's body 130. Through implementation of the present cuffed fluid line extraction device 01, tissue ingrowth cuff 150 and fluid line 140 extraction is capable of being performed in a more expeditious manner while dramatically reducing the risk of severing or puncturing the fluid line 140, and at the same time reducing the physical trauma, in the form of excessive bleeding, bruising, scar tissue formation, scaring, or physical pain, sustained by the patient.

Those skilled in the art will appreciate the present invention may be used for the extraction of varied and numerous types of tissue ingrowth cuffs 150 and fluid lines 140 from both human and non-human bodies in different embodiments. By way of example, these fluid lines 140 can be used in the introduction of antibiotics, parenteral nutrition, IV fluids, blood products, peritoneal dialysis, and chemotherapy treatment; obtaining of blood samples; and preparation for plasmapheresis and hemodialysis.

The cuffed fluid line extraction device 01 illustratively includes a cylindrical-shaped grip member 10, at the proximal end of the device and a beveled, “C-shaped,” smooth cutting head 30 at the distal end. In the present example, the exterior of the cuffed fluid line extraction device 01 grip member 10 has rigid gripping and knuckling exterior structures 110, 110′ to facilitate the medical practitioner's ability to grip and manipulate the cuffed fluid line extraction device 01 by hand or instrument. The exterior gripping and knuckling structures 110, 110′ allow the medical practitioner to effectively and easily rotate the cuffed fluid line extraction device about the longitudinal axis 160 360° in both clockwise and counter-clockwise directions to circumferentially cut and separate the ingrown cuff 150 and fluid line 140 from the surrounding tissue.

The grip member 10 extends from its proximal end of the cuffed fluid line extraction device 01 towards the distal end of the grip member 10, which is spaced apart from the proximal end and terminating at the grip member and cutting shaft retaining means 40, here the cutting shaft member 20 is affixed into the interior surface of the grip member 10 by force fit, frictional or other retention means. The cutting shaft member 20 has a proximal end closest to the grip member 10 and a distal end spaced apart from the proximal end and terminating in a beveled, “C-shaped,” smooth cutting head 30 as shown in FIG. 1. The cutting shaft member 20 is comprised of three separate and distinct connective support members 50 that are equidistantly spaced and extend from the proximal end of the cutting shaft member 20 to the beveled, “C-shaped,” smooth cutting head 30 located at the distal end of the cutting shaft member 20.

The beveled, “C-shaped,” smooth cutting head 30 at the distal end of the cuffed fluid line extraction device 01 and cutting shaft member 20 is comprised of a smooth and sharp beveled cutting head blade 120, 120′. There are two separate and distinct embodiments of the beveled cutting head blade 120, 120′ their use being depending on the extraction being performed by the medical practitioner. One embodiment is a beveled cutting head blade that tapers inwardly 120; the other embodiment has a beveled cutting head blade tapering outwardly 120′ from the cuffed fluid line extraction device 01. Both embodiments aid in the separation of tissue from the tissue ingrowth cuff 140 and the fluid line 150. These beveled cutting head blades 120,120′ permit the cuffed fluid line extraction device 01 to be inserted into the patient's body 130, around the fluid line 140 and tissue ingrowth cuff 150. The beveled, “C-shaped,” smooth cutting head 30 located at the distal end of the cuffed fluid line extraction device 01 will preferably have a circumference greater than that of the tissue ingrowth cuff 150 to allow the entire tissue ingrowth cuff 150 to be removed with the fluid line 140. As those skilled in the art will appreciate, there will be varying beveled, “C-shaped,” smooth cutting head 30 circumferences to correspond to the varying tissue ingrowth cuff 150 dimensions. When inserted into the patient's body 130, through rotational application of pressure, the cuffed fluid line extraction device 01, separates tissue from both the ingrown cuff 150 and the fluid line 140 utilizing the beveled cutting head blades 120,120′.

The cuffed fluid line extraction device 01 further comprises a small fluid line removal channel 90; the channel extends from the proximal end of the cuffed fluid line extraction device 01, through the grip member 10, cutting shaft member 20, and beveled, C-shaped,” smooth cutting head 30. The fluid line removal channel 90 terminates at the distal end of the cuffed fluid line extraction device 01 that is separated apart from the proximal end, at the beveled, “C-shaped,” smooth cutting edge head 30. The fluid line removal channel 90 and opening slit 80 shall have openings that are at least slightly greater than the diameter of the fluid line 140 for insertion of the fluid line 140 through the opening slit 80 and receipt of the fluid line 140 in the fluid line removal channel 90. As those skilled in the art will appreciate, there will be varying fluid line removal channel 90 and opening slit 80 widths to correspond to the varying fluid line 140 circumferential dimensions. In the present example, the cuffed fluid line extraction device 01 further comprises a cuffed fluid line channel guide 100, within the fluid line removal channel 90 for support and retention of the fluid line 140 during the manipulation and rotation of the cuffed fluid line extraction device 01. The cuffed fluid line channel guide 100 spans the length of the fluid line removal channel 90 as seen in FIG. 1. The fluid line 140 rests against the cuffed fluid line channel guide 100 and is retained in place by the grip member 10, cutting shaft member 20, and beveled, “C-shaped,” smooth cutting head 30 being relatively encapsulated with the exception of the fluid line removal channel 90 and opening slit 80. Maintaining of the fluid line 140 within the fluid line removal channel 90 and against the cuffed fluid line channel guide 100 allows for the beveled, “C-shaped,” smooth cutting head 30 to remain centered on the tissue ingrown cuff 150 and the fluid line 140 when penetrating the patient's body 130. In addition to the cuffed fluid line channel guide shown 100, those skilled in the art will understand the ability of the cuffed fluid line channel guide 100 to take other shapes or orientations.

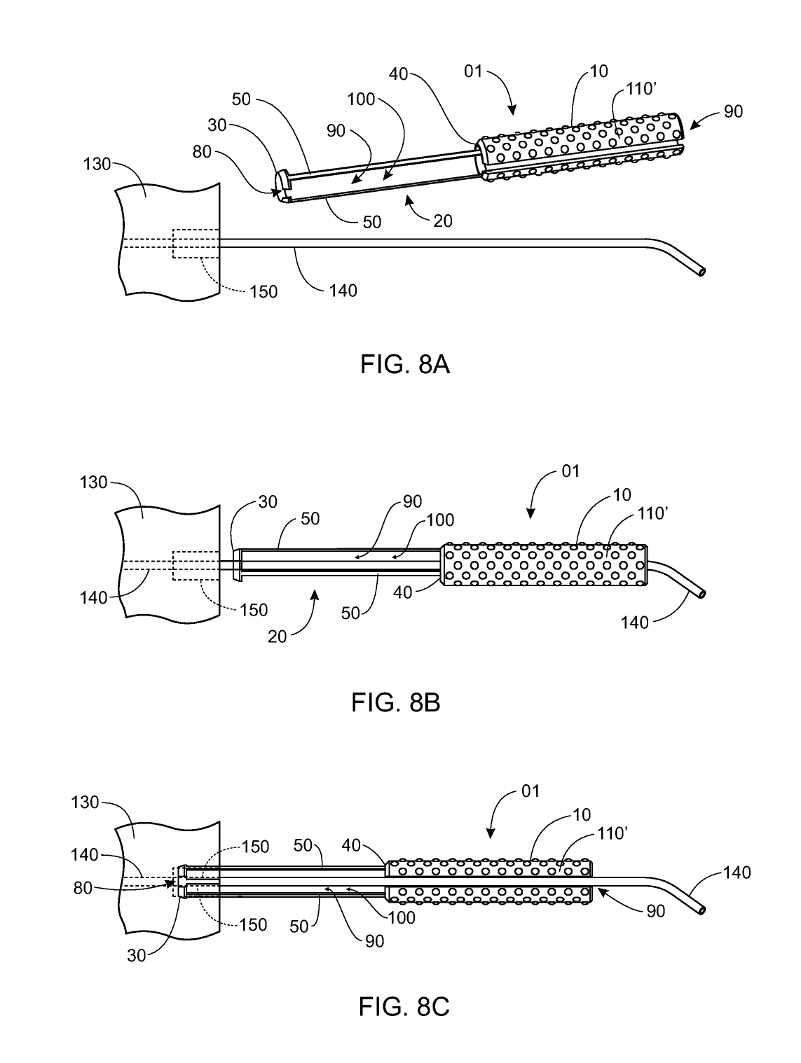

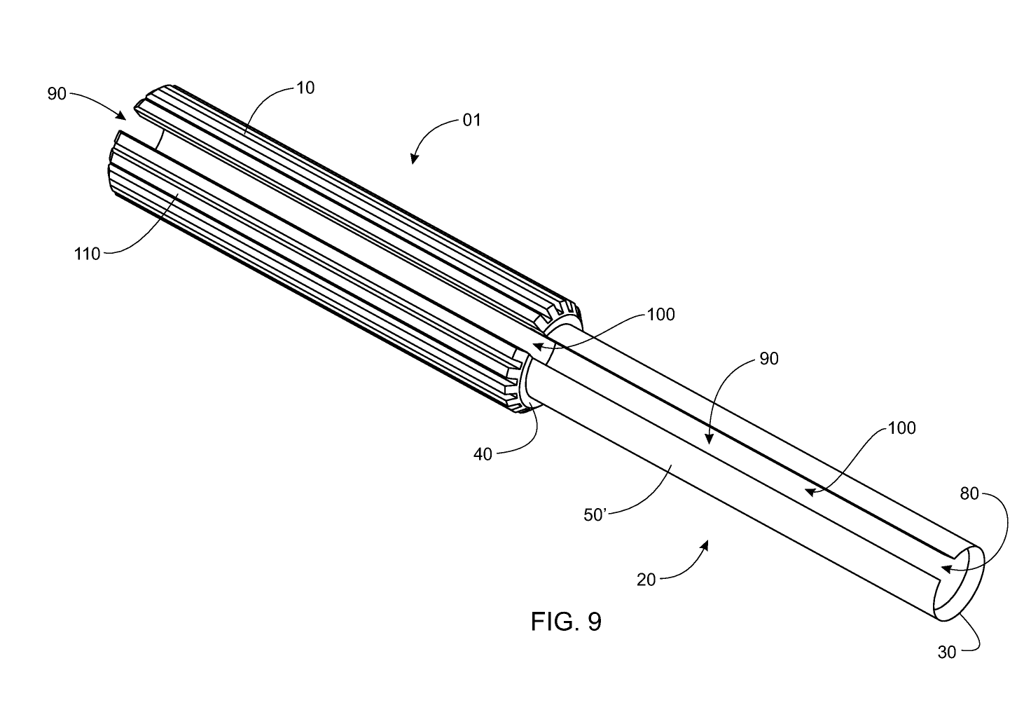

As shown in FIGS. 8A and 8B, it is preferred that the fluid line 140 first be inserted through the opening slit 80 and into the fluid line removal channel 90 of the cuffed fluid line extraction device 01, assuring that the fluid line 140 travels against the cuffed fluid line channel guide 100 from the distal end through and out of the end of the grip member 10 at the proximal end. Next, the cuffed fluid line extraction device 01 proceeds down the fluid line 140 towards the patient's body 130. When the beveled, “C-shaped,” smooth cutting head 30 located at the distal end of the cuffed fluid line extraction device 01 makes contact with the exterior surface of the patient's body 130, it is preferred that the cuffed fluid line extraction device 01 be perpendicular in relation to the exterior of the patient's body 130.

Once the cuffed fluid line extraction device 01 has penetrated the patient's body shown in FIG. 8C, it is then manipulated by the medical practitioner by hand or instrument, in concert with application of some pressure in the direction of the patient's body 130. This manipulation may be performed in many ways; however, it is preferred that rotational movement of the cuffed fluid line extraction device 01 is made in the clock-wise and counter-clockwise directions. The rotational movement of the cuffed fluid line extraction device 01 may consist of several full 360° rotations, in either the clock-wise or counter-clockwise direction, around the fluid line 140 and/or tissue ingrowth cuff 150, and/or rotations in the clockwise or counter-clockwise direction that are less than 360° around the fluid line 140 and/or ingrowth cuff 150. Using the applied pressure and rotational circular motion, the sharp and beveled cutting head blades 120, 120′ of the “C-shaped,” smooth cutting head 30 cuts, dissects and separates the tissue surrounding the tissue ingrowth cuff 150, while reducing the risk of the severing or puncture of the fluid line 140 by the beveled cutting head blades 120,120′. This allows the beveled, “C-shaped,” smooth cutting head 30 to penetrate further into the patient's body 130 and around the tissue ingrowth cuff 150 and/or fluid line 140 as seen in FIG. 8C. Once the beveled, “C-shaped,” smooth cutting head 30 cuts, dissects and separates the body tissue surrounding the tissue ingrowth cuff 150 and/or the fluid line 140 and is around the tissue ingrowth cuff 150, the fluid line 140 and the tissue ingrowth cuff 150, are extracted together from the patient's body 130.

The cuffed fluid line extraction device 01 is preferably made out of bio-compatible material such as bio-compatible metal or plastic for the cutting shaft member 20, cutting shaft connective support sections 50, 50′, beveled, “C-shaped,” smooth cutting head 30, and beveled cutting head blades 120, 120′, and grip member 10. It is particularly advantageous to construct the cuffed fluid line extraction device 01 from bio-compatible plastic where appropriate in embodiments where the device 01 is intended for one use or a very limited number of uses.

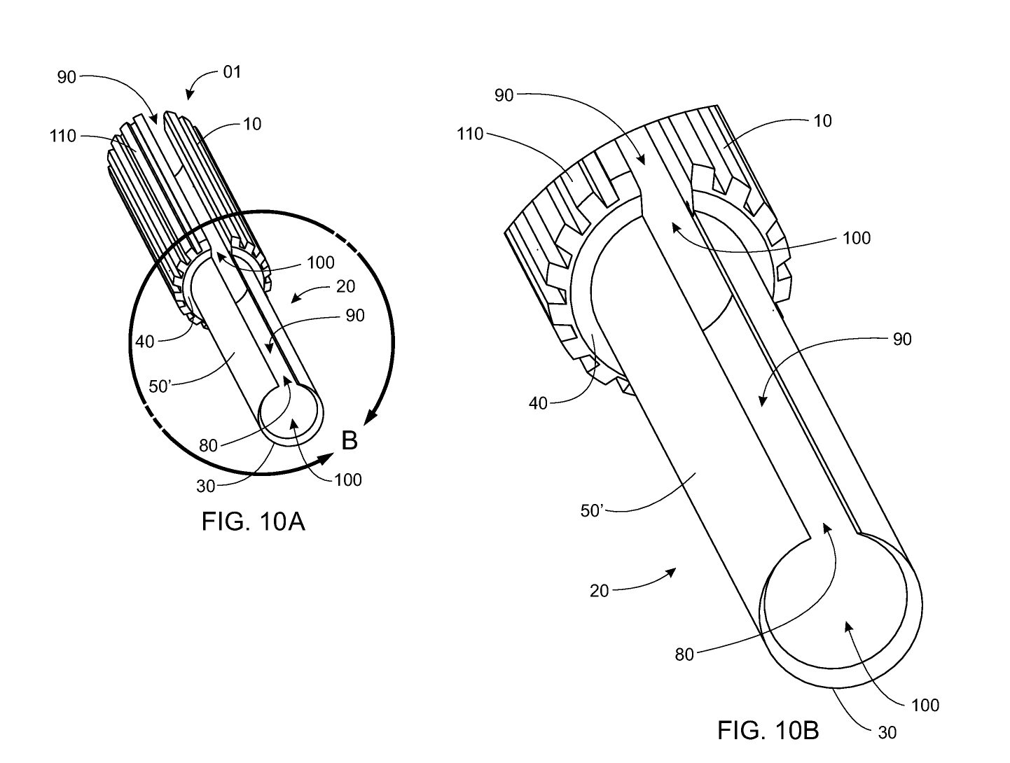

Turning to FIGS. 9 and 10, an alternate embodiment, for the cutting shaft member 20, is shown, comprised of a single, solid, partially enclosed, cutting section connective support member 50′, extending from the distal end of the grip member 10 and retaining means 40 to the beveled, “C-shaped,” smooth cutting head 30 to provide additional structural support.

While the preferred embodiments have been shown and described a particular configuration of cuffed fluid line extraction devices and methods for removing those devices, such embodiments are exemplary. While certain embodiments and details have been included herein for purposes of illustrating aspects of the present invention, it will be apparent to those skilled in the art that various changes in the systems, devices, and methods disclosed herein may be made without departing from the scope of the present disclosure, which is defined, in part, in the appended claims. It is understood that there is no intent to limit the invention by such disclosure, but rather, it is intended to cover all modifications and alternate constructions falling within the spirit and scope of this invention. The words “including” and “having,” as used herein, including the claims, shall have the same meaning as the word “comprising.”

What which is claimed is:

1. A cuffed fluid line extraction device for extraction of tissue ingrowth cuffs and fluid lines from a body comprising:
a grip member extending from the proximal end of the cuffed fluid line extraction device towards the distal end of the grip including a removal channel for receipt of a fluid line tube, an opening slit for insertion of the fluid line tube, a means for retaining a cutting shaft member formed integrally on the distal end of said grip member; and
a cutting shaft member comprising proximal and distal ends, extending uniformly from the said means of retaining, said proximal end of the cutting shaft member being retained by said retaining means, said cutting shaft member distal end extending from the proximal end and terminating in a razor sharp “C-shaped,” smooth cutting head, said cutting shaft member and “C-shaped,” smooth cutting head including a removal channel for receipt of a fluid line tube and an opening slit for insertion of the fluid line tube.

2. The fluid line extraction device of claim 1, wherein said retaining means includes a receptacle dimensioned to receive said cutting shaft member.

3. The cuffed fluid line extraction device of claim 1, wherein said proximal end of the cutting shaft member is force fit and frictionally retained within said retaining means.

4. The cuffed fluid line extraction device for extraction of claim 1 wherein the fluid line has a cuff which has ingrown into the surrounding subcutaneous tissue and wherein the “C-shaped,” smooth cutting head at the distal end of the cutting shaft member has circumference greater than that of the tissue ingrowth cuff.

5. The cuffed fluid line extraction device of claim 1 wherein the “C-shaped,” smooth cutting head, cutting shaft member and grip member of the device defines an opening slit for insertion of the fluid line tube in the cuffed fluid line removal channel.

6. The cuffed fluid line extraction device of claim 1 wherein said cutting shaft member comprises three equally spaced support members connecting said cutting shaft member proximal end to the “C-shaped,” smooth cutting head located at the cutting shaft member distal end and spanning the cuffed fluid line removal channel.

7. The cuffed fluid line extraction device of claim 1 wherein said cutting shaft member comprises a single, solid shaft connecting said cutting shaft member proximal end to the “C-shaped,” smooth cutting head located at the cutting shaft member distal end and spanning the cuffed fluid line removal channel.

8. The cuffed fluid line extraction device of claim 1 wherein the “C-shaped,” smooth cutting head at distal end at of said cutting shaft member can bevel inward or outward.

9. The cuffed fluid line extraction device claim 1 wherein said grip member comprises a smooth surface on the interior surface thereof.

10. The cuffed fluid line extraction device of claim 9 wherein said grip member consists of rigid griping or knuckled structures on the exterior surface to facilitate manipulation and twisting in clock-wise or counter clock-wise directions by hand or instrument for cutting away subcutaneous tissue away from the ingrowth tissue cuff and fluid line.

11. The cuffed fluid line removal device of claim 1 wherein the fluid line comprises a catheter having an ingrowth tissue cuff carried thereon, and wherein the “C-shaped,” smooth cutting head has a circumference greater than the circumference of the tissue ingrowth cuff.

12. A method for removing a cuffed fluid line from a body comprising:
providing a cuffed fluid line removal device comprising a grip member with griping or knuckled exterior structures, a cutting shaft member, and a “C-shaped,” smooth cutting head, the distal end spaced apart from the proximal end, a cuffed fluid line removal channel extending from the “C-shaped,” smooth cutting edge at the distal end through the cutting shaft member and terminating at proximal end of the grip for receiving the fluid line, with the device also serving as a guide carried within the fluid line removal channel;
inserting the cuffed fluid line tube through an opening slit extending from the “C-shaped,” smooth cutting head at the distal end to the grip member proximal end into the cuffed fluid line removal channel so that the guide supports the fluid line tube;
manipulating and rotating of the device by hand or by instrument, while pushing downward on the grip member so that the razor sharp “C-shaped,” smooth cutting head cuts and dissects body subcutaneous tissue away from the fluid line; and
removal the cuffed fluid line from the body.

13. The method for removing a cuffed fluid line from a body of claim 12 wherein the cuffed fluid line comprises a catheter having a tissue ingrowth cuff carried thereon; wherein the distal end of the fluid line extraction device at the “C-shaped,” smooth cutting head has a circumference greater than a circumference of the tissue ingrowth cuff adhered to the cuffed fluid line, and wherein removal includes manipulation of the grip member by twisting or rotating in the clock-wise or counter clock-wise directions by hand or by instrument so that the razor sharp “C-shaped,” smooth cutting head cuts and dissects subcutaneous body tissue away from the adhered tissue ingrowth cuff.

14. The method for removing a cuffed fluid line from a body of claim 12 wherein the “C-shaped,” smooth cutting head defines an opening slit therein, and wherein receipt of the cuffed fluid line includes insertion of the cuffed fluid line tube in the cuffed fluid line removal channel through said opening slit.

15. The method for removing a cuffed fluid line from a body of claim 12 wherein the cuffed fluid line tube guide extends from the “C-shaped,” smooth cutting head through the cutting shaft member, terminating at the proximal end of the grip member and spanning the entire fluid line removal channel.

16. The method for removing a cuffed fluid line from a body of claim 12 wherein said cutting shaft member comprises three equally spaced support members connecting said cutting shaft member proximal end to the “C-shaped” smooth cutting head located at the cutting shaft member distal end and spanning the cuffed fluid line removal channel and cuffed fluid line tube guide.

17. The method for removing a cuffed fluid line from a body of claim 12 wherein said cutting shaft member comprises a single, solid shaft connecting said cutting shaft member proximal end to the “C-shaped” smooth cutting head located at the cutting shaft member distal end and spanning the cuffed fluid line removal channel and cuffed fluid line tube guide.

18. The fluid line removal device of claim 12 wherein the distal end of the device defines a razor sharp “C-shaped,” smooth cutting head.
